(19) United States
(12) Patent Application Publication
(10) Pub. No.: US 2012/0041079 A1
(43) Pub. Date: Feb. 16, 2012

(54) DEXTRAN FUNCTIONALIZED BY HYDROPHOBIC AMINO ACIDS

(75) Inventors: Gerard SOULA, Meyzieu (FR); Olivier Soula, Meyzieu (FR); Remi Soula, Meyzieu (FR); Richard Charvet, Rillieux La Pape (FR)

(73) Assignee: ADOCIA, Lyon (FR)

(21) Appl. No.: 13/250,803

(22) Filed: Sep. 30, 2011

Related U.S. Application Data
(63) Continuation-in-part of application No. 12/078,441, filed on Mar. 31, 2008.
Application No. 12/078,441 is a continuation-in-part of application No. PCT/IB2007/002807, filed on Sep. 26, 2007.
Application No. PCT/IB2007/002807 is a continuation of application No. PCT/IB2006/002666, filed on Sep. 26, 2006.
(60) Provisional application No. 60/907,376, filed on Mar. 29, 2007.

(30) Foreign Application Priority Data
Mar. 29, 2007 (FR) 07 02316

(51) Int. Cl. A61K 47/36; C07H 15/18; C07H 15/04; C07H 17/02

(52) U.S. Cl. 514/777; 536/51

(57) ABSTRACT

Dextran of the following general formula I:

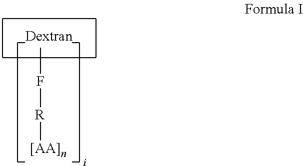

is described, wherein
R represents a chain containing from 1 to 15 carbon atoms and having at least one acid functional group prior to attachment to AA, the chain optionally being branched and/or unsaturated and containing one or more heteroatoms, such as O, N or/and S,
F represents an ester, a carbamate or an ether,
AA represents a hydrophobic amino acid radical, L or D, derived from a coupling between an amine of an amino acid and at least one acid functional group of R; the amino acid, prior to attachment to R, being selected from the group consisting of tryptophan, phenylalanine, leucine, isoleucine, alanine and valine, and alcohol, amide or decarboxylated derivatives thereof, and alkaline cation salts thereof,
i represents the molar fraction of substituent F—R-[AA]n per glycosidic unit and is from 0.1 to 2,
n represents the molar fraction of R groups substituted by AA and is from 0.05 to 1,
   and wherein for R groups not substituted by AA, the acid(s) of the group R are alkaline cation carboxylates such as Na or K.

The present application is a continuation-in-part application of U.S. application Ser. No. 12/078,441 filed Mar. 31, 2008, which in turn is a continuation-in-part application of PCT/IB2007/002807 filed Sep. 26, 2007, which claims the benefit of PCT/IB2006/002666 filed Sep. 26, 2006 and French Application No. 07 02316 filed Mar. 29, 2007 and U.S. Provisional Application No. 60/907,376 filed Mar. 29, 2007. The disclosures of the prior applications is hereby incorporated by reference herein in their entireties.

The present invention relates to novel biocompatible polysaccharides based on dextran.

These polysaccharides can be used especially for the administration of active ingredient(s) (AI) to humans or to animals for therapeutic and/or prophylactic purposes.

The present invention relates to novel amphiphilic dextran derivatives functionalized by at least one hydrophobic alpha-amino acid. These novel dextran derivatives have good biocompatibility, and their hydrophobicity can readily be modulated without altering their biocompatibility.

Among the amphiphilic dextrans, the carboxymethyl dextrans from Biodex described in U.S. Pat. No. 6,646,120 are modified by benzylamine. This hydrophobic group does not belong to the family of the alpha-amino acids.

Dellacherie et al. have also described amphiphilic dextrans (Durand, A. et al., Biomacromolecules 2006, 7, 958-964)(Durand, Alain et al., Colloid Polym. Sci. 2006, 284, 536-545) obtained by reaction of the hydroxyl functional groups of the dextran with epoxides (phenyl glycidyl ether, 1,2-epoxyoctane or 1,2-epoxydodecane). The described amphiphilic polysaccharides are therefore not functionalized by amino acids.

In U.S. Pat. No. 5,750,678, Bauer et al. describe dextrans functionalized by C10 to C14 fatty acids. The resulting polysaccharides are amphiphilic but are not modified by hydrophobic amino acids.

A recent review of dextran-based functional polysaccharides (Heinze, Thomas et al., Adv Polym Sci 2006, 205, 199-291) does not take into account dextran functionalized by a hydrophobic amino acid.

Accordingly, the invention relates to a dextran functionalized by at least one hydrophobic alpha-amino acid radical, designated AA, said alpha-amino acid in the form of an alkaline cation grafted or bonded to the dextran by a bonding arm R and a functional group F,
R represents a chain containing from 1 to 15 carbon atoms and having at least one acid functional group prior to attachment to AA, the chain optionally being branched and/or unsaturated and containing one or more heteroatoms, such as O, N or/and S,
F represents an ester, a carbamate or an ether,
AA represents a hydrophobic amino acid radical, L or D, derived from a coupling between an amine of an amino acid and at least one acid functional group of R; the amino acid, prior to attachment to R, being selected from the group consisting of tryptophan, phenylalanine, leucine, isoleucine and valine, and alcohol, amide or decarboxylated derivatives thereof, and alkaline cation salts thereof.

A hydrophobic amino acid radical is understood as representing the product of coupling between the amine of an amino acid and an acid carried by the group R.

According to the invention, the functionalized dextran corresponds to the following general formula:

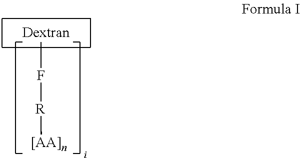

### Wherein

R represents a chain containing from 1 to 15 carbon atoms and having at least one acid functional group prior to attachment to AA, the chain optionally being branched and/or unsaturated and containing one or more heteroatoms, such as O, N or/and S,
F represents an ester, a carbamate or an ether,
AA represents a hydrophobic amino acid radical, L or D, derived from a coupling between an amine of an amino acid and at least one acid functional group of R; the amino acid, prior to attachment to R, being selected from the group consisting of tryptophan, phenylalanine, leucine, isoleucine, alanine and valine, and alcohol, amide or decarboxylated derivatives thereof, and alkaline cation salts thereof
i represents the molar fraction of substituent F—R-[AA]n per glycosidic unit and is from 0.1 to 2,
n represents the molar fraction of R groups substituted by AA and is from 0.05 to 1.

When R is not substituted by AA, the acid(s) of the group R are alkaline cation carboxylates, preferably such as Na, K.

In an embodiment, F is an ester a carbamate or an ether.

In an embodiment, the polysaccharide before the grafting of the amino acid radical is a carboxymethyl dextran (DMC) of formula IV

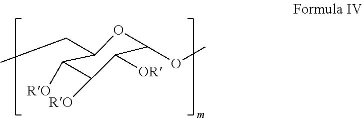

Wherein R′ is

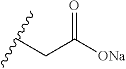

or the corresponding acid or —H

In an embodiment, the polysaccharide before the grafting of the amino acid radical is a dextran monosuccinic ester or succinic acid dextran (DSA) of formula IV

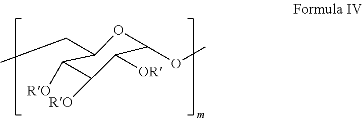

Wherein R′ is

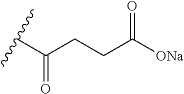

or the corresponding acid or —H

In an embodiment, the polysaccharide according to the invention is characterized in that the group F—R before the grafting of the amino acid is selected from the following groups:

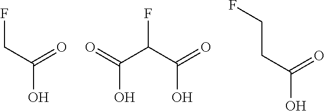

or their alkaline cation salts.

In an embodiment, the dextran according to the invention is characterized in that the hydrophobic amino acid is selected from tryptophan derivatives, such as tryptophan, tryptophanol, tryptophanamide, 2-indole ethylamine and their alkaline cation salts.

In an embodiment, the dextran according to the invention is characterized in that the hydrophobic amino acid is selected from phenylalanine and its alcohol, amide and its alkaline cation salt.

In an embodiment, the dextran according to the invention is characterized in that the hydrophobic amino acid is selected from leucine, isoleucine, alanine and valine and their alcohol, amide and their alkaline cations salts.

The dextran has a degree of polymerization m comprised between 10 to 10,000.

In an embodiment, it has a degree of polymerization m comprised between 10 to 1000.

In another embodiment, it has a degree of polymerization m comprised between 10 to 500.

The dextrans according to the invention can be obtained by grafting an ester of the amino acid in question onto the dextran modified by a group R, the ester group being finally hydrolyzed under alkaline conditions (NaOH, 0.01N).

In an embodiment, an ester of formula II

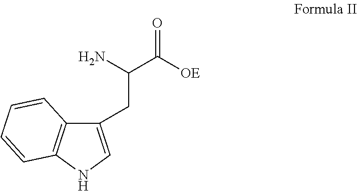

E being represents a group which can be:
a linear or branched C₁- to C₄-alkyl,
is grafted onto a dextran (DMC) of formula IV

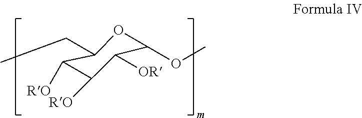

Wherein R′ is

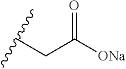

or the corresponding acid or —H
and then under alkaline conditions the Na salt of formula VI is obtained.

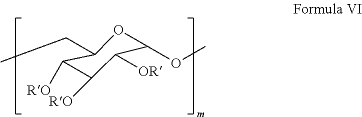

Wherein R′ is

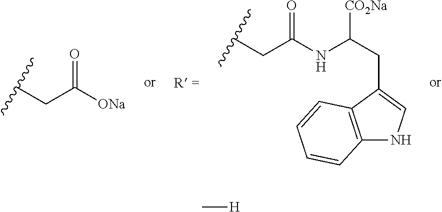

In an embodiment, a commercial ester of phenylalanine such as the methyl ester or the ethyl ester is grafted onto a sodium methylcarboxylate dextran (DMC) of formula formula IV

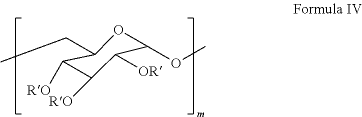

Wherein R′ is

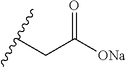

or the corresponding acid or —H and then under alkaline conditions the Na salt of formula VII is obtained.

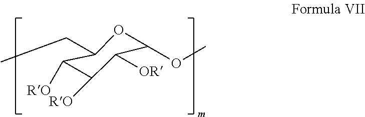

Wherein R′ is

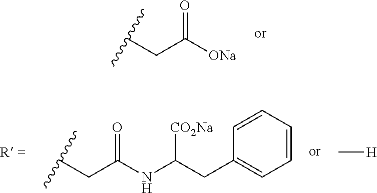

In another embodiment the dextrans according to the invention can be obtained by directly grafting the amino acid onto the dextran of formula IV

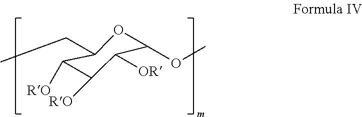

Wherein R′ is

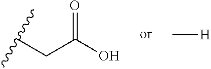

in DMF in the presence of N-Methylmorpholine (NMM) and ethylchloroformate (EtOCOCl).

In another embodiment the dextrans according to the invention are obtained by directly grafting tryptophan onto the dextran the dextran of formula IV

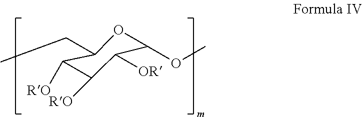

Wherein R′ is

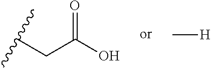

in DMF in the presence of N-Methylmorpholine (NMM) and ethylchloroformate (EtOCOCl).

In another embodiment the dextrans according to the invention can be obtained by directly grafting the amino acid onto the dextran of formula IV

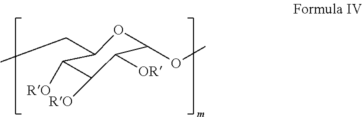

Wherein R′ is

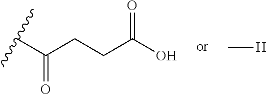

in DMF in the presence of N-Methylmorpholine (NMM) and ethylchloroformate (EtOCOCl).

In another embodiment the dextrans according to the invention are obtained by directly grafting tryptophan onto the dextran of formula IV

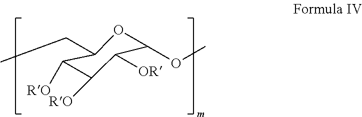

Wherein R′ is

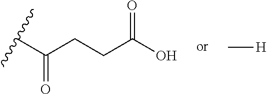

in DMF in the presence of N-Methylmorpholine (NMM) and ethylchloroformate (EtOCOCl).

The invention relates also to a dextran chosen amongst the following dextrans:

For each dextran a name is given with between brackets a short name with number that are the (DS) given which accounts for the average number of each substituent regarding the dextran and not relative to the acid groups.

The degree of substitution (DS) represents the average number of substituted hydroxyls per mono- or disaccharide unit of the dextran chain. This number represents an average of the analytical determination and thus, for a given dextran, it can be theoretically any number from 0 to k, k being the total number of hydroxyls on each mono- or disaccharide unit. Since there are k reactive hydroxyls per glucose unit, the maximum DS value is k when the dextran is totally substituted.
sodium methylcarboxylate dextran functionalized with the sodium salt of phenylalanine, DP=10, i=1.69 and n=0.64 (10DMC(1.69)PheONa(1.08)).
sodium methylcarboxylate dextran functionalized with the sodium salt of phenylalanine, DP=10, i=1.06 and n=0.51 (10DMC(1.06)PheONa(0.54)).
sodium methylcarboxylate dextran functionalized with the sodium salt of isoleucine, DP=5, i=1.08 and n=0.32 (5DMC(1.08)IleONa(0.35)).
sodium methylcarboxylate dextran functionalized with the sodium salt of leucine, DP=10, i=1.06 and n=0.33 (10DMC(1.06)LeuONa(0.35)).
sodium methylcarboxylate dextran functionalized with the sodium salt of valine, DP=10, i=1.06 and n=0.42 (10DMC(1.06)ValONa(0.45)).
sodium methylcarboxylate dextran functionalized with the sodium salt of phenylalanine, DP=5, i=1.65 and n=0.39 (5DMC(1.65)PheONa(0.65)).
sodium methylcarboxylate dextran functionalized with the sodium salt of phenylalanine, DP=5, i=1.08 and n=0.42 (5DMC(1.08)PheONa(0.45)).
sodium methylcarboxylate dextran functionalized with the sodium salt of phenylalanine, DP=5, i=2.1 and n=0.48 (5DMC(2.1)PheONa(1.0)).
sodium methylcarboxylate dextran functionalized with the sodium salt of phenylalanine, DP=10, i=1.65 and n=0.39 (10DMC(1.65)PheONa(0.65)).
sodium methylcarboxylate dextran functionalized with the sodium salt of phenylalanine, DP=10, i=1.06 and n=0.42 (10DMC(1.06)PheONa(0.45)).
sodium methylcarboxylate dextran functionalized with the sodium salt of tryptophan, DP=5, i=1.65 and n=0.27 (5DMC(1.65)TrpONa(0.45)).
sodium methylcarboxylate dextran functionalized with the sodium salt of tryptophan, DP=5, i=1.65 and n=0.61 (5DMC(1.65)TrpONa(1.0)).
sodium methylcarboxylate dextran functionalized with the sodium salt of tryptophan, DP=40, i=1.04 and n=0.45 (40DMC(1.04)TrpONa(0.45)).
sodium succinate dextran functionalized with the sodium salt of tryptophan, DP=5, i=1.7 and n=0.76 5DSA(1.7)TrpONa(1.3)).
sodium N-methylcarboxylate dextran urethane functionalized with the sodium salt of phenylalanine, DP=5, i=1.8 and n=0.36 (5DUGly(1.8)PheONa(0.65)).
sodium N-methylcarboxylate dextran urethane functionalized with the sodium salt of phenylalanine, DP=5, i=1.01 and n=0.50 (5DUGly(1.01)PheONa(0.50)).

The invention relates also to a pharmaceutical composition comprising one of the dextrans according to the invention as described hereinbefore, and at least one active ingredient.

Active ingredient is understood as being a product in the form of a single chemical entity or in the form of a combination having physiological activity. Said active ingredient can be exogenous, that is to say it is supplied by the composition according to the invention. It can also be endogenous, for example the growth factors which will be secreted in a wound during the first phase of scarring and which it will be possible to retain on said wound by means of the composition according to the invention.

The invention relates also to a pharmaceutical composition according to the invention as described hereinbefore, characterized in that it is administrable by the oral, nasal, vaginal, buccal route.

The invention relates also to a pharmaceutical composition according to the invention as described hereinbefore, characterized in that it is obtained by drying and/or lyophilization.

The invention relates also to a pharmaceutical composition according to the invention as described hereinbefore, characterized in that it is administrable in the form of a stent, a film or a coating of implantable biomaterials, in the form of an implant.

The invention relates also to a pharmaceutical composition according to the invention as described hereinbefore, characterized in that the active ingredient is selected from the group constituted by proteins, glycoproteins, peptides and non-peptide therapeutic molecules.

The possible pharmaceutical compositions are either in liquid form or in the form of a powder, an implant or a film.

In the case of local and systemic release, the possible modes of administration are by the intravenous, subcutaneous, intradermal, intramuscular, oral, nasal, vaginal, ocular, buccal route, etc.

The invention relates also to the use of the functionalized dextrans according to the invention in the preparation of pharmaceutical compositions as described hereinbefore.

### EXAMPLES OF SYNTHESES OF POLYSACCHARIDES

For each polysaccharide a name is given with between brackets a short name with number that are the DS given which accounts for the average number of each substituent regarding the dextran and not relative to the acid groups.

### Polysaccharide 1: Synthesis of a Sodium Methylcarboxylate Dextran Functionalized With the Sodium Salt of Tryptophan (40DMC(1.04)TrpONa(0.45))

8 g (148 mmol of hydroxyl functions) of dextran of weight average molecular weight 40 kg/mol (Pharmacosmos, degree of polymerization of 176) and NaBH₄ (11 mg; 0.3 mmol), are dissolved in water (420 g/L). To this solution is added NaOH 10 N (15 ml; 148 mmol NaOH). The mixture is heated at 35° C. and sodium chloroacetate (23 g; 198 mmol) is added. The mixture is progressively heated to 60° C. and this temperature is maintained 100 additional minutes. The mixture is diluted with water, neutralized with acetic acid and purified by ultrafiltration on a 5 kDa cut-off PES membrane against water. The final solution concentration is determined by dry solid content, and an acido-basic titration in water/12cetone 50/50 (V/V) is performed to determine the methylcarboxylate substitution degree.

Solid dry content: [polysaccharide]=31.5 mg/g

According to the acido-basic titration, the methylcarboxylate substitution degree is 1.04 per sugar unit.

The sodium methylcarboxylate dextran solution is acidified over an anionic Purolite resin which leads to the corresponding methylcarboxylic acid dextran which is then freeze dried over 18 hours.

10 g of methylcarboxylic acid dextran (47 mmol of methylcarboxylic acid functions) are dissolved in DMF (62 g/L). The solution is cooled to 0° C. and NMM (5.24 g; 52 mmol) and EtOCOCl (5.6 g; 52 mmol) are added. After 10 minutes, L-tryptophan (Ajinomoto) (4.1 g; 20 mmol) is added and the mixture is stirred at 10° C. An aqueous imidazole solution (340 g/L) is added and the mixture is heated to 30° C. 50 ml of water are added and the obtained solution is purified by ultrafiltration on a 10 kDa cut-off PES membrane against NaCl 0.9%, NaOH 0.01N, NaCl 0.9% and water. The final solution concentration is determined by dry solid content. A solution sample is freeze dried and analyzed by ¹H NMR in D₂O to determine the molar fraction of acid groups grafted with the sodium salt of tryptophan.

Dry solid content: [Polysaccharide 1]=39.8 mg/g

According to ¹H NMR: the molar fraction of acid groups functionalized with the sodium salt of tryptophan is 0.45.

### Polysaccharide 2: Synthesis of a Sodium Methylcarboxylate Dextran Functionalized With the Sodium Salt of Tryptophan (5DMC(1.65)TrpONa(1.0))

A sodium methylcarboxylate dextran characterized by a methylcarboxylate substitution degree of 1.04 per sugar unit, is synthesized from dextran of weight average molecular weight 5 kg/mol (Pharmacosmos, degree of polymerization of 19) according to a process similar to the one used for polysaccharide 1 synthesis, and freeze dried.

8 g (64 mmol of hydroxyl functions) of sodium methylcarboxylate dextran characterized by a methylcarboxylate substitution degree of 1.04 are dissolved in water (1000 g/L). To this solution is added NaOH 10 N (6 ml; 64 mmol). The mixture is heated at 35° C. and sodium chloroacetate (7.6 g; 65 mmol) is added. The mixture is progressively heated to 60° C. and this temperature is maintained 100 additional minutes. The mixture is diluted with water, neutralized with acetic acid and purified by ultrafiltration on a 5 kDa cut-off PES membrane against water. The final solution concentration is determined by dry solid content, and an acido-basic titration in water/acetone 50/50 (V/V) is performed to determine the methylcarboxylate substitution degree.

Solid dry content: [polysaccharide]=45.8 mg/g

According to the acido-basic titration, the methylcarboxylate substitution degree is 1.65 per sugar unit.

The sodium methylcarboxylate dextran solution is acidified over an anionic Purolite resin which leads to the corresponding methylcarboxylic acid dextran which is then freeze dried over 18 hours.

10 g of methylcarboxylic acid dextran (64 mmol of methylcarboxylic acid functions) are dissolved in DMF (62 g/L). The solution is cooled to 0° C. and NMM (7.1 g; 70 mmol) and EtOCOCl (7.6 g; 70 mmol) are added. After 10 minutes, L-tryptophan (Ajinomoto) (11.9 g; 58 mmol) is added and the mixture is stirred at 10° C. An aqueous imidazole solution (340 g/L) is added and the mixture is heated to 30° C. 70 ml of water are added the obtained solution is purified by ultrafiltration on a 5 kDa cut-off PES membrane against NaCl 0.9%, NaOH 0.01N, NaCl 0.9% and water. The final solution concentration is determined by dry solid content. A solution sample is freeze dried and analyzed by ¹H NMR in D₂O in order to determine the molar fraction of acid groups grafted with the sodium salt of tryptophan.

Dry solid content: [Polysaccharide 2]=37.2 mg/g

According to ¹H NMR: the molar fraction of acid groups functionalized with the sodium salt of tryptophan is 0.61.

Polysaccharide 3: Synthesis of a Sodium Methylcarboxylate Dextran Functionalized with the Sodium Salt of Tryptophan (5DMC(1.65)TrpONa(0.45))

A methylcarboxylic acid dextran characterized by a methylcarboxylic acid substitution degree of 1.65 per sugar unit, is synthesized from dextran of weight average molecular weight 5 kg/mol (Pharmacosmos, degree of polymerization of 19) according to a process similar to the one used for polysaccharide 2 synthesis, and freeze dried. Polysaccharide 3 is a sodium methyl carboxylate dextran functionalized with the sodium salt of tryptophan synthesized according to a process similar to the one used for polysaccharide 1 synthesis.

Dry solid content: [Polysaccharide 3]=28.5 mg/g

According to ¹H NMR: the molar fraction of acid groups functionalized with the sodium salt of tryptophan is 0.27.

Polysaccharide 4: Synthesis of a Sodium Methylcarboxylate Dextran Functionalized with the Sodium Salt of Phenylalanine (10DMC(1.06)PheONa(0.45)

A methylcarboxylic acid dextran characterized by a methylcarboxylic acid substitution degree of 1.06 per sugar unit, is synthesized from dextran of weight average molecular weight 10 kg/mol (Pharmacosmos, degree of polymerization of 39) according to a process similar to the one used for polysaccharide 1 synthesis, and freeze dried.

10 g of methylcarboxylic acid dextran (47 mmol of methylcarboxylic acid functions) are dissolved in DMF (79 g/L) and cooled to 0° C. A mixture of hydrochloride salt of phenylalanine ethyl ester (Bachem) (4.6 g; 20 mmol) in DMF is prepared (100 g/L). Triethylamine (2.0 g; 20 mmol) is added to this mixture. Once the polysaccharide solution reaches 0° C., NMM (4.8 g; 47 mmol) and EtOCOCl (5.1 g; 47 mmol) are added. After 10 minutes, the solution of phenylalanine ethyl ester is added and the mixture is stirred at 10° C. An aqueous imidazole solution (340 g/L) is added and the mixture is heated to 30° C. 50 ml of water are added and the obtained solution is purified by ultrafiltration on a 10 kDa cut-off PES membrane against NaCl 0.9%, NaOH 0.1N, NaCl 0.9% and water. The final solution concentration is determined by dry solid content. A solution sample is freeze dried and analyzed by ¹H NMR in D₂O to determine the molar fraction of acid groups grafted with the sodium salt of phenylalanine.

Dry solid content: [Polysaccharide 4]=31.8 mg/g

According to ¹H NMR: the molar fraction of acid groups functionalized with the sodium salt of phenylalanine is 0.42.

Polysaccharide 5: Synthesis of a Sodium Methylcarboxylate Dextran Functionalized with the Sodium Salt of Phenylalanine (10DMC(1.65)PheONa(0.65))

A methylcarboxylic acid dextran characterized by a methylcarboxylic acid substitution degree of 1.65 per sugar unit, is synthesized from dextran of weight average molecular weight 10 kg/mol (Pharmacosmos, degree of polymerization of 39) according to a process similar to the one used for polysaccharide 2 synthesis, and freeze dried.

10 g of methylcarboxylic acid dextran (64 mmol of methylcarboxylic acid functions) are dissolved in DMF (59 g/L) and cooled to 0° C. A mixture of hydrochloride salt of phenylalanine ethyl ester (Bachem) (6.2 g; 27 mmol) in DMF is prepared (100 g/L). Triethylamine (2.7 g; 27 mmol) is added to this mixture. Once the polysaccharide solution reaches 0° C., NMM (6.5 g; 64 mmol) and EtOCOCl (6.9 g; 64 mmol) are added. After 10 minutes, the solution of phenylalanine ethyl ester is added and the mixture is stirred at 10° C. An aqueous imidazole solution (340 g/L) is added and the mixture is heated to 30° C. 70 ml of water are added and the obtained solution is purified by ultrafiltration on a 10 kDa cut-off PES membrane against NaOH 0.1N, NaCl 0.9% and water. The final solution concentration is determined by dry solid content. A solution sample is freeze dried and analyzed by ¹H NMR in D₂O to determine the molar fraction of acid groups grafted with the sodium salt of phenylalanine.

Dry solid content: [Polysaccharide 5]=40.9 mg/g

According to ¹H NMR: the molar fraction of acid groups functionalized with the sodium salt of phenylalanine is 0.39.

Polysaccharide 6: Synthesis of a Sodium Methylcarboxylate Dextran Functionalized with the Sodium Salt of Phenylalanine (5DMC(2.1)PheONa(1.0))

A methylcarboxylic acid dextran characterized by a methylcarboxylate substitution degree of 1.65 per sugar unit, is synthesized from dextran of weight average molecular weight 5 kg/mol (Pharmacosmos, degree of polymerization of 19) according to a process similar to the one used for polysaccharide 2 synthesis, and freeze dried. 8 g (37 mmol of hydroxyl functions) of sodium methylcarboxylate dextran characterized by methylcarboxylate substitution degree of 1.65 are dissolved in water (530 g/L). The solution is heated at 65° C. and sodium chloroacetate (11.1 g; 95 mmol) is added. NaOH 10N (14 ml; 136 mmol) is added dropwise and the mixture is further stirred at 65° C. The mixture is diluted with water, neutralized with acetic acid and purified by ultrafiltration on a 5 kDa cut-off PES membrane against water. The final solution concentration is determined by dry solid content, and an acido-basic titration in water/16cetone 50/50 (V/V) is performed to determine the methylcarboxylate substitution degree.

Dry solid content: [Polysaccharide]=25.8 mg/g

According to the acido-basic titration, the methylcarboxylate substitution degree is 2.1 per sugar unit.

The sodium methylcarboxylate dextran solution is acidified over an anionic Purolite resin which leads to the corresponding methylcarboxylic acid dextran which is then freeze dried over 18 hours.

Polysaccharide 6 is a sodium methylcarboxylate dextran functionalized with the sodium salt of phenylalanine synthesized according to a process similar to the one used for polysaccharide 5 synthesis.

Dry solid content: [Polysaccharide 6]=17.7 mg/g

According to ¹H NMR: the molar fraction of acid groups functionalized with the sodium salt of phenylalanine is 0.48.

Polysaccharide 7: Synthesis of a Sodium Methylcarboxylate Dextran Functionalized with the Sodium Salt of Phenylalanine (5DMC(1.08)PheONa(0.45))

A methylcarboxylic acid dextran characterized by a methylcarboxylic acid substitution degree of 1.08 per sugar unit, is synthesized from dextran of weight average molecular weight 5 kg/mol (Pharmacosmos, degree of polymerization of 19) according to a process similar to the one used for polysaccharide 1 synthesis, and freeze dried. Polysaccharide 7 is a sodium methylcarboxylate dextran functionalized with the sodium salt of phenylalanine synthesized according to a process similar to the one used for polysaccharide 4 synthesis.

Dry solid content: [Polysaccharide 7]=28.8 mg/g

According to ¹H NMR: the molar fraction of acid groups functionalized with the sodium salt of phenylalanine is 0.42.

Polysaccharide 8: Synthesis of a Sodium Methylcarboxylate Dextran Functionalized with the Sodium Salt of Phenylalanine (5DMC(1.65)PheONa(0.65))

A methylcarboxylic acid dextran characterized by a methylcarboxylic acid substitution degree of 1.65 per sugar unit, is synthesized from dextran of weight average molecular weight 5 kg/mol (Pharmacosmos, degree of polymerization of 19) according to a process similar to the one used for polysaccharide 2 synthesis, and freeze dried. Polysaccharide 8 is a sodium methylcarboxylate dextran functionalized with the sodium salt of phenylalanine synthesized according to a process similar to the one used for polysaccharide 5 synthesis.

Dry solid content: [Polysaccharide 8]=42.1 mg/g

According to ¹H NMR: the molar fraction of acid groups functionalized with the sodium salt of phenylalanine is 0.39.

Polysaccharide 9: Synthesis of a Sodium Methylcarboxylate Dextran Functionalized with the Sodium Salt of Valine (10DMC(1.06)ValONa(0.45))

A methylcarboxylic acid dextran characterized by a methylcarboxylic acid substitution degree of 1.06 per sugar unit, is synthesized from dextran of weight average molecular weight 10 kg/mol (Pharmacosmos, degree of polymerization of 39) according to a process similar to the one used for polysaccharide 1 synthesis, and freeze dried.

Polysaccharide 9 is a sodium methylcarboxylate dextran functionalized with the sodium salt of valine synthesized according to a process similar to the one used for polysaccharide 4 synthesis with the hydrochloride salt of valine ethyl ester (Bachem) as graft.

Dry solid content: [Polysaccharide 9]=33.3 mg/g

According to ¹H NMR: the molar fraction of acid groups functionalized with the sodium salt of valine is 0.42.

Polysaccharide 10: Synthesis of a Sodium Methylcarboxylate Dextran Functionalized with the Sodium Salt of Leucine (10DMC(1.06)LeuONa(0.35))

A methylcarboxylic acid dextran characterized by a methylcarboxylic acid substitution degree of 1.06 per sugar unit, is synthesized from dextran of weight average molecular weight 10 kg/mol (Pharmacosmos, degree of polymerization of 39) according to a process similar to the one involved in polysaccharide 1 synthesis, and freeze dried.

Polysaccharide 10 is a sodium methylcarboxylate dextran functionalized with the sodium salt of leucine synthesized according to a process similar to the one used for polysaccharide 4 synthesis with the hydrochloride salt of leucine ethyl ester (Bachem) as graft.

Dry solid content: [Polysaccharide 10]=23.3 mg/g

According to ¹H NMR: the molar fraction of acid groups functionalized with the sodium salt of leucine is 0.33.

Polysaccharide 11: Synthesis of a Sodium Methylcarboxylate Dextran Functionalized with the Sodium Salt of Isoleucine (5DMC(1.08)IleONa(0.35))

A methylcarboxylic acid dextran characterized by a methylcarboxylic acid substitution degree of 1.08 per sugar unit, is synthesized from dextran of weight average molecular weight 5 kg/mol (Pharmacosmos, degree of polymerization of 19) according to a process similar to the one involved in polysaccharide 1 synthesis, and freeze dried.

Polysaccharide 11 is a sodium methylcarboxylate dextran functionalized with the sodium salt of isoleucine synthesized according to a process similar to the one used for polysaccharide 4 synthesis with the hydrochloride salt of isoleucine methyl ester (Bachem) as graft.

Dry solid content: [Polysaccharide 11]=27.7 mg/g

According to ¹H NMR: the molar fraction of acid groups functionalized with the sodium salt of isoleucine is 0.32.

Polysaccharide 12: Synthesis of a Sodium Methylcarboxylate Dextran Functionalized with the Sodium Salt of Phenylalanine (10DMC(1.06)PheONa(0.54))

A methylcarboxylic acid dextran characterized by a methylcarboxylic acid substitution degree of 1.06 per sugar unit, is synthesized from dextran of weight average molecular weight 10 kg/mol (Pharmacosmos, degree of polymerization of 38) according to a process similar to the one used for polysaccharide 1 synthesis, and freeze dried.

Polysaccharide 12 is a sodium methylcarboxylate dextran functionalized with the sodium salt of phenylalanine synthesized according to a process similar to the one used for polysaccharide 4 synthesis.

Dry solid content: [Polysaccharide 12]=31.8 mg/g

According to ¹H NMR: the molar fraction of acid groups functionalized with the sodium salt of phenylalanine is 0.51.

Polysaccharide 13: Synthesis of a Sodium Methylcarboxylate Dextran Functionalized with the Sodium Salt of Phenylalanine (10DMC(1.69)PheONa(1.08))

A methylcarboxylic acid dextran characterized by a methylcarboxylic acid substitution degree of 1.65 per sugar unit, is synthesized from dextran of weight average molecular weight 10 kg/mol (Pharmacosmos, degree of polymerization of 38) according to a process similar to the one used for polysaccharide 2 synthesis, and freeze dried.

Polysaccharide 13 is a sodium methyl carboxylate dextran functionalized with the sodium salt of phenylalanine synthesized according to a process similar to the one used for polysaccharide 5 synthesis.

Dry solid content: [Polysaccharide 13]=40.9 mg/g

According to ¹H NMR: the molar fraction of acid groups functionalized with the sodium salt of phenylalanine is 0.64.

Polysaccharide 14: Synthesis of a Sodium Succinate Dextran Functionalized with the Sodium Salt of Tryptophan (5DSA(1,7)TrpONa(1,3))

A sodium succinate dextran is obtained from dextran of weight average molecular weight 5 kg/mol (Pharmacosmos, degree of polymerization of 19) according to the procedure described by Sanchez-Chaves et al. in Polymer 1998, 39(18), 2751-2757. The final solution concentration is determined by dry solid content, and an acido-basic titration in water/acetone 50/50 (V/V) is performed to determine the succinate substitution degree.

Solid dry content: [polymer]=31.5 mg/g

According to the acido-basic titration, the succinate substitution degree is 1.73 per sugar unit.

The sodium succinate dextran solution is acidified over an anionic Purolite resin which leads to the corresponding succinic acid dextran which is then freeze dried over 18 hours.

Polysaccharide 14 is a sodium succinate dextran functionalized with the sodium salt of tryptophan synthesized according to a process similar to the one used for polysaccharide 2 synthesis.

Dry solid content: [Polymer 14]=32.4 mg/g

According to ¹H NMR: the molar fraction of acid groups functionalized with the sodium salt of tryptophan is 0.76.

Polymer 15: Synthesis of a Sodium N-Methylcarboxylate Dextran Urethane Functionalized with the Sodium Salt of Phenylalanine (5DUGly(1.8)PheONa(0.65))

8 g (148 mmol of hydroxyl functions) of dextran of weight average molecular weight 5 kg/mol (Pharmacosmos, degree of polymerization of 19), are dissolved in a DMF/DMSO mixture at 60° C. NaBH₄ (192 mg; 5 mmol) is added and the mixture is stirred at 60° C. DABCO (1,4-Diazabicyclo[2.2.2]octane, 2.2 g; 20 mmol) and toluene (7 mL) are then added. The mixture is distilled under reduced pressure at 120° C. Once the mixture temperature reaches 80° C. on cooling, ethyl ester isocyanatoacetate (Aldrich) (19.1 g; 148 mmol) is added and the solution is stirred at this temperature. The mixture is then poured into NaOH 0.1N and the polymer solution is purified by ultrafiltration on a 5 kDa cut-off PES membrane against NaOH 0.1N, NaCl 0.9% and water. The final solution concentration is determined by dry solid content, and an acido-basic titration in water/acetone 50/50 (V/V) is performed to determine the N-methylcarboxylate substitution degree.

Solid dry content: [polymer]=20.2 mg/g

According to the acido-basic titration, the N-methylcarboxylate substitution degree is 1.82 per sugar unit.

The sodium N-methylcarboxylate dextran urethane solution is acidified over an anionic Purolite resin which leads to the corresponding N-methylcarboxylic acid dextran urethane which is then freeze dried over 18 hours.

Polysaccharide 15 is a sodium N-methylcarboxylate dextran urethane functionalized with the sodium salt of phenylalanine synthesized according to a process similar to the one used for polysaccharide 4 synthesis.

Dry solid content: [Polymer 15]=33.1 mg/g

According to ¹H NMR: the molar fraction of acid groups functionalized with the sodium salt of phenylalanine is 0.36.

Polymer 16: Synthesis of a Sodium N-Methylcarboxylate Dextran Urethane Functionalized with the Sodium Salt of Phenylalanine (5DUGly(1.01)PheONa(0.50)).

A N-methylcarboxylic acid dextran urethane characterized by a methylcarboxylic acid substitution degree of 1.01 per sugar unit, is synthesized from dextran of weight average molecular weight 5 kg/mol (Pharmacosmos, degree of polymerization of 19) according to a process similar to the one used for polysaccharide 15 synthesis, and freeze dried.

Polysaccharide 16 is a sodium N-methylcarboxylate dextran urethane functionalized with the sodium salt of phenylalanine synthesized according to a process similar to the one used for polysaccharide 15 synthesis.

Dry solid content: [Polymer 16]=27.6 mg/g

According to ¹H NMR: the molar fraction of acid groups functionalized with the sodium salt of phenylalanine is 0.50.

Polymer 16: Synthesis of a Sodium N-Methylcarboxylate Dextran Urethane Functionalized with the Sodium Salt of Phenylalanine (5DUGly(1.01)PheONa(0.50)).

A N-methylcarboxylic acid dextran urethane characterized by a methylcarboxylic acid substitution degree of 1.01 per sugar unit, is synthesized from dextran of weight average molecular weight 5 kg/mol (Pharmacosmos, degree of polymerization of 19) according to a process similar to the one used for polysaccharide 15 synthesis, and freeze dried.

Polysaccharide 16 is a sodium N-methylcarboxylate dextran urethane functionalized with the sodium salt of phenylalanine synthesized according to a process similar to the one used for polysaccharide 15 synthesis.

Dry solid content: [Polymer 16]=27.6 mg/g

According to ¹H NMR: the molar fraction of acid groups functionalized with the sodium salt of phenylalanine is 0.50.

### Examples of Interaction Between a Protein and the Dextrans of the Invention

### Insulin Solubility at its Isoelelectric Point

Human insulin has an isoelectric point, pI, of 5.3. As a result, human insulin precipitates out of aqueous solution when the pH is equal to the pI, at 5.3.

The solubility of human insulin in presence of the dextrans of the invention at pH 5.3 was investigated.

An aqueous solution of human insulin at 200 UI/mL is prepared at pH 7. An aqueous solution of the dextrans of the invention at 20 mg/mL is prepared at pH 7. The polymer solution is added to the insulin solution (50/50 v/v mixture) to lead to a solution at 100 UI/mL of human insulin and at 10 mg/mL of polymer. The pH of each solution is decreased to pH 5.3 by addition of 200 mM acetic acid.

The solubility of human insulin is documented by the aspect of the solution. If the solution is turbid, human insulin is insoluble at its isoelectric point. If the solution is clear, human insulin is soluble at its isoelectric point. In the case of an aqueous human insulin solution at 100 UI/mL at pH 7 being acidified to pH 5.3 with acetic acid, the solution obtained is turbid as expected.

| Polysaccharide | Solubility of human insulin |
|---|---|
| Polysaccharide 2 | Yes |
| Polysaccharide 3 | Yes |
| Polysaccharide 4 | Yes |
| Polysaccharide 5 | Yes |
| Polysaccharide 6 | Yes |
| Polysaccharide 7 | Yes |
| Polysaccharide 8 | Yes |
| Polysaccharide 9 | Yes |
| Polysaccharide 10 | Yes |
| Polysaccharide 11 | Yes |
| Methylcarboxylate Dextran from | No |
| example 2 | |
| Methylcarboxylate Dextran from | No |
| example 4 | |

The solubility of human insulin at pH 5.3 in presence of the dextrans of the invention demonstrates that there is an interaction between these polymers and the human insulin. The polymers are covering the surface of insulin and prevent its aggregation at its pI. On the contrary, the methylcarboxylate dextrans before grafting of the hydrophobic amino acid do not prevent the precipitation of human insulin at its pI.

What is claimed is:

1. Dextran of the following general formula I: 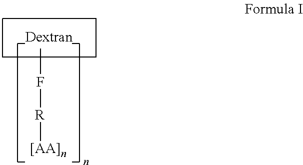
wherein
R represents a chain containing from 1 to 15 carbon atoms and having at least one acid functional group prior to attachment to AA, the chain optionally being branched and/or unsaturated and containing one or more heteroatoms, such as O, N or/and S,
F represents an ester, a carbamate or an ether,
AA represents a hydrophobic amino acid radical, L or D, derived from a coupling between an amine of an amino acid and at least one acid functional group of R; the amino acid, prior to attachment to R, being selected from the group consisting of tryptophan, phenylalanine, leucine, isoleucine, alanine and valine, and alcohol, amide or decarboxylated derivatives thereof, and alkaline cation salts thereof,
i represents the molar fraction of substituent F—R-[AA]n per glycosidic unit and is from 0.1 to 2,
n represents the molar fraction of R groups substituted by AA and is from 0.05 to 1,
and wherein for R groups not substituted by AA, the acid(s) of the group R are alkaline cation carboxylates, preferably Na, K.

2. Dextran according to claim 1, wherein the group F is an ester a carbamate or an ether.

3. Dextran according to claim 1, wherein the hydrophobic amino acid is selected from tryptophan derivatives, such as tryptophan, tryptophanol, tryptophanamide, 2-indole ethylamine and their alkaline cation salts.

4. Dextran according to claim 1, wherein the hydrophobic amino acid is selected from phenylalanine and its alcohol, amide and its alkaline cation salt.

5. Dextran according to claim 1, wherein the hydrophobic amino acid is selected from leucine, isoleucine, alanine and valine and their alcohol, amide and their alkaline cations salts.

6. Dextran according to claim 1, wherein the dextran has a degree of polymerization between 10 to 10,000.

7. Dextran according to claim 1, wherein the dextran is selected from the group:
sodium methylcarboxylate dextran functionalized with the sodium salt of phenylalanine, DP=10, i=1.69 and n=0.64 (10DMC(1.69)PheONa(1.08)),
sodium methylcarboxylate dextran functionalized with the sodium salt of phenylalanine, DP=10, i=1.06 and n=0.51 (10DMC(1.06)PheONa(0.54)),
sodium methylcarboxylate dextran functionalized with the sodium salt of isoleucine, DP=5, i=1.08 and n=0.32 (5DMC(1.08)IleONa(0.35)),
sodium methylcarboxylate dextran functionalized with the sodium salt of leucine, DP=10, i=1.06 and n=0.33 (10DMC(1.06)LeuONa(0.35)),
sodium methylcarboxylate dextran functionalized with the sodium salt of valine, DP=10, i=1.06 and n=0.42 (10DMC(1.06)ValONa(0.45)),
sodium methylcarboxylate dextran functionalized with the sodium salt of phenylalanine, DP=5, i=1.65 and n=0.39 (5DMC(1.65)PheONa(0.65)),
sodium methylcarboxylate dextran functionalized with the sodium salt of phenylalanine, DP=5, i=1.08 and n=0.42 (5DMC(1.08)PheONa(0.45)),
sodium methylcarboxylate dextran functionalized with the sodium salt of phenylalanine, DP=5, i=2.1 and n=0.48 (5DMC(2.1)PheONa(1.0)),
sodium methylcarboxylate dextran functionalized with the sodium salt of phenylalanine, DP=10, i=1.65 and n=0.39 (10DMC(1.65)PheONa(0.65)),
sodium methylcarboxylate dextran functionalized with the sodium salt of phenylalanine, DP=10, i=1.06 and n=0.42 (10DMC(1.06)PheONa(0.45)),
sodium methylcarboxylate dextran functionalized with the sodium salt of tryptophan, DP=5, i=1.65 and n=0.27 (5DMC(1.65)TrpONa(0.45)),
sodium methylcarboxylate dextran functionalized with the sodium salt of tryptophan, DP=5, i=1.65 and n=0.61 (5DMC(1.65)TrpONa(1.0)),
sodium methylcarboxylate dextran functionalized with the sodium salt of tryptophan, DP=40, i=1.04 and n=0.45 (40DMC(1.04)TrpONa(0.45)),
sodium succinate dextran functionalized with the sodium salt of tryptophan, DP=5, i=1.7 and n=0.76 5DSA(1.7)TrpONa(1.3)),
sodium N-methylcarboxylate dextran urethane functionalized with the sodium salt of phenylalanine, DP=5, i=1.8 and n=0.36 (5DUGly(1.8)PheONa(0.65)), and
sodium N-methylcarboxylate dextran urethane functionalized with the sodium salt of phenylalanine, DP=5, i=1.01 and n=0.50 (5DUGly(1.01)PheONa(0.50)).

8. Pharmaceutical composition comprising at least one dextran according to claim 1 and at least one active ingredient.
